(19) United States
(12) Patent Application Publication
(10) Pub. No.: US 2026/0242533 A1
(43) Pub. Date: Aug. 20, 2026

(54) POLYURETHANE FOAM FOR COSMETIC COMPOSITION IMPREGNATION AND METHOD OF MANUFACTURING THE SAME

(71) Applicant: Kolmar Korea Co., Ltd., Sejong-si (KR)

(72) Inventors: Hee Ho PARK, Seoul (KR); Gi Min Tak, Seoul (KR); Yong Woo Kim, Seoul (KR); Ji Hyun Lee, Seoul (KR)

(21) Appl. No.: 19/536,125

(22) Filed: Feb. 10, 2026

(30) Foreign Application Priority Data
Feb. 14, 2025 (KR) 10-2025-0019669

(51) Int. Cl. C08G 18/76; C08G 18/48; C08G 18/50; C08G 18/63

(52) U.S. Cl. CPC C08G 18/7664; C08G 18/4829; C08G 18/5021; C08G 18/632; C08G 18/7621; C08G 2110/0083

(57) ABSTRACT

A polyurethane foam for cosmetic composition impregnation and application of the composition to a surface such as human skin is provided. The polyurethane foam for cosmetic composition impregnation can be formed by mixing a polyether polyol and a polyester polyol in a weight ratio of 1:0.17 to 1:0.33 and foaming the mixture. The provided polyurethane foam for cosmetic composition impregnation has improved long-term stability, can be easily filled and loaded with a cosmetic composition, and can easily discharge the cosmetic composition upon application of pressure.

### CROSS REFERENCE TO RELATED APPLICATION

This application claims priority from and the benefit of Korean Patent Application No. 10-2025-0019669, filed on Feb. 14, 2025, which is hereby incorporated by reference for all purposes as if fully set forth herein.

### BACKGROUND

### Field

Embodiments of the invention relate generally to a polyurethane foam for cosmetic composition impregnation and a method of manufacturing the same.

### Discussion of the Background

In the modern cosmetics market, impregnation members such as sponges are widely used to load and then apply to the skin color cosmetics such as foundation, which serves as a base for other cosmetics to follow. Various materials can function as the impregnation member, and polyurethane foam is being increasingly used due to its suitable porosity, elasticity, light weight, and the like.

Polyurethane is synthesized by chemically reacting polyols and diisocyanates. Here, polyols for manufacturing polyurethane are classified into polyether-based polyols and polyester-based polyols. Polyurethane manufactured using a polyester-based polyol has the advantage of withstanding the repeated pressure and friction that it is subjected to when cosmetics are applied, due to its excellent physical durability as indicated by its tensile strength, abrasion resistance, tear resistance, and the like. On the other hand, polyurethane manufactured using a polyether-based polyol has the advantage of maintaining its shape and function even in a highly moist environment because it has excellent moisture resistance. There is a need to appropriately combine the advantages of polyurethane manufactured using a polyester-based polyol and the advantages of polyurethane manufactured using a polyether-based polyol and to manufacture polyurethane foams having physical properties that are optimized for their use as impregnation members and for the convenient delivery of cosmetic compositions to surfaces.

The above information disclosed in this Background section is only for understanding of the background of the inventive concepts, and, therefore, it may contain information that does not constitute prior art.

### SUMMARY

The present application is directed to providing a polyurethane foam for cosmetic composition impregnation and a method of manufacturing the same.

Additional features of the inventive concepts will be set forth in the description which follows, and in part will be apparent from the description, or may be learned by practice of the inventive concepts.

According to an embodiment of the present application, there is provided a polyurethane foam for cosmetic composition impregnation. The polyurethane foam for cosmetic composition impregnation may be formed by mixing a polyether polyol and a polyester polyol in a weight ratio of 1:0.17 to 1:0.33 and foaming the mixture.

The polyurethane foam for cosmetic composition impregnation may have a pore number per linear inch of 70 to 80 ppi and a hardness of 70 to 85 on the Asker F hardness scale as measured by the Asker durometer Type F.

The polyurethane foam for cosmetic composition impregnation may have a tensile strength of 220 to 240 kPa and an elongation rate of 210 to 250%.

The polyurethane foam for cosmetic composition impregnation may be impregnated with a cosmetic composition having an oil-phase ingredient content of 25 wt % to 50 wt %.

The polyurethane foam for cosmetic composition impregnation may be impregnated with a cosmetic composition having a viscosity of 7,000 cps or more and 45,000 cps or less.

According to another embodiment of the present application, there is provided a method of manufacturing polyurethane foam for cosmetic composition impregnation. The method of manufacturing polyurethane foam for cosmetic composition impregnation includes mixing a composition for manufacturing polyurethane foam, the composition including a polyether polyol and a polyester polyol; and foaming the composition, wherein the polyether polyol and the polyester polyol are mixed in a weight ratio of 1:0.17 to 1:0.33 in the composition for manufacturing polyurethane foam.

The polyurethane foam for cosmetic composition impregnation may have a pore number per linear inch of 70 to 80 ppi and a hardness of 70 to 85 on the Asker F hardness scale as measured by the Asker durometer Type F.

The polyurethane foam for cosmetic composition impregnation may have a tensile strength of 220 to 240 kPa and an elongation rate of 210 to 250%.

The method of manufacturing polyurethane foam for cosmetic composition impregnation may further comprise impregnating the polyurethane foam for cosmetic composition impregnation with a cosmetic composition having an oil-phase ingredient content of 25 wt % to 50 wt %.

The method of manufacturing polyurethane foam for cosmetic composition impregnation may further comprise impregnating the polyurethane foam for cosmetic composition impregnation with a cosmetic composition having a viscosity of 7,000 cps or more and 45,000 cps or less.

The composition for manufacturing polyurethane foam may include the polyether polyol, the polyester polyol, a foaming agent, a catalyst, and an additive.

The mixing step of the method for manufacturing polyurethane foam may be performed at a temperature of 18 to 28° C. and a stirring speed of 4,000 to 5,000 rpm for 20 to 40 minutes.

In addition, the foaming may be performed at a discharge pressure of 35,000 to 45,000 Pa.

It is to be understood that both the foregoing general description and the following detailed description are exemplary and explanatory and are intended to provide further explanation of the invention as claimed.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 shows, for Manufacturing Examples 1 to 3, photographs taken after each polyurethane foam was pressed with a sharp object for one second after three months in an incubator at 70° C. and 90% humidity;

FIG. 2 shows a) a photograph of the polyurethane foam of Manufacturing Example 1 filled with a cosmetic composition having an oil-phase ingredient content of 40 wt % and a viscosity of 20,000 cps and b) a photograph of a cosmetic puff to which the cosmetic composition was applied by pressing the filled polyurethane foam of (a); and

FIG. 3 shows a) a photograph of the polyurethane foam of Manufacturing Example 1 filled with a cosmetic composition having an oil-phase ingredient content of 20 wt % and a viscosity of 50,000 cps and b) a photograph of a cosmetic puff to which the cosmetic composition was applied by pressing the filled polyurethane foam of (a).

### DETAILED DESCRIPTION

In the following description, for the purposes of explanation, numerous specific details are set forth in order to provide a thorough understanding of various embodiments or implementations of the invention. As used herein “embodiments” and “implementations” are interchangeable words that are non-limiting examples of devices or methods employing one or more of the inventive concepts disclosed herein. It is apparent, however, that various embodiments may be practiced without these specific details or with one or more equivalent arrangements. In other instances, well-known structures and devices are shown in block diagram form in order to avoid unnecessarily obscuring various embodiments. Further, various embodiments may be different, but do not have to be exclusive. For example, specific shapes, configurations, and characteristics of an embodiment may be used or implemented in another embodiment without departing from the inventive concepts.

Unless otherwise specified, the illustrated embodiments are to be understood as providing features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z-axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the exemplary term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various embodiments are described herein with reference to sectional and/or exploded illustrations that are schematic illustrations of idealized embodiments and/or intermediate structures. As such, variations from the shapes of the illustrations as a result, for example, of manufacturing techniques and/or tolerances, are to be expected. Thus, embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

In terms of the physical properties described in the present specification, when a temperature at the time of measurement affects the physical properties, unless otherwise specified, the physical properties are those measured at room temperature and normal pressure.

In terms of the physical properties described in the present specification, unless otherwise specified, the physical properties may basically follow the SI unit system. For example, weight may be expressed in kilograms (kg), and length may be expressed in meters (m).

The term “room temperature” used in the present specification refers to a natural temperature in an unheated and uncooled state and may be, for example, any temperature ranging from 10° C. to 30° C. such as about 25° C. Unless otherwise specified in the present specification, the unit of temperature is degrees Celsius (° C.).

The term “normal pressure” used in the present specification refers to a natural pressure in a state where the pressure is not increased and decreased and is generally atmospheric pressure ranging from about 700 mmHg to 800 mmHg.

The phrase “a to b” used in the present specification refers to a range of a to b including a and b. For example, including a to b parts by weight is the same as including a range of a to b parts by weight.

Throughout the present specification, the values expressed in range format should be flexibly interpreted to include not only the numerical values explicitly stated as limits of such a range, but also all of the individual numerical values or subranges included in such a range, as if each numerical value and subrange are explicitly stated. For example, a range of “about 0.1% to about 5%” or “about 0.1% to 5%” should be interpreted to include not only about 0.1% to about 5%, but also individual values (e.g., 1%, 2%, 3%, and 4%) and subranges (e.g., 0.1% to 0.5%, 1.1% to 2.2%, and 3.3% to 4.4%) within the indicated range.

The term “about” or “approximately” used in the present specification means that dimensions, sizes, formulations, parameters, shapes, and other quantities and characteristics may not be exact, and as necessary, may be approximate and/or larger or smaller by reflecting tolerances, conversion factors, rounding, measurement errors, and other factors known to those skilled in the art. Generally, regardless of whether or not explicitly stated, the term “about” or “approximately” is applied to any dimension, size, formulation, parameter, shape, or other quantities or characteristics described in the present specification. The term “about” used in the present specification can allow for a degree of variability in a predetermined value or range, for example, within 10%, 5%, or 1% of the stated value or the stated range limit, and includes the stated exact value or range.

When a method for measuring or evaluating a specific dimension, size, formulation, parameter, shape, and other quantities and characteristics is not described in the present specification, those skilled in the art can easily recognize a conventionally known method for measuring or evaluating the same or can easily derive the measurement or evaluation method by referring to a known technology known prior to the filing date of the present specification.

In the present specification, when a portion “includes” an element, another element may be further included, rather than excluding the other element, unless otherwise described.

The term “or” used in the present application is intended to mean an inclusive “or” rather than an exclusive “or.” In other words, unless otherwise specified or clear from context, “X uses A or B” is intended to mean one of the natural inclusive permutations. For the case in which X uses A; X uses B; or X uses both A and B, “X uses A or B” may be applied to any of the above cases. In addition, the term “and/or” used in the present specification should be understood to refer to and include all possible combinations of one or more of the listed relevant configurations.

According to one aspect of the present application, there is provided a polyurethane foam for cosmetic composition impregnation. Polyurethane foam is a material synthesized by chemically reacting a polyol and a diisocyanate, and is suitable as the material of a member for cosmetic composition impregnation because it has appropriate porosity, elasticity, light weight, and the like. The polyol is a polymer with a plurality of hydroxyl groups (—OH), and the diisocyanate is a compound with two isocyanate groups (—NCO).

In an embodiment, the polyurethane foam for cosmetic composition impregnation may be formed by foaming a composition for manufacturing polyurethane foam. Here, the composition for manufacturing polyurethane foam may include a polyether polyol and a polyester polyol. In other words, the polyurethane foam for cosmetic composition impregnation may be formed by mixing a polyether polyol and a polyester polyol and foaming the mixture. The polyether polyol is a polyol with repeating ether bonds (—O—) and may include, for example, polypropylene glycol (PPG), polyethylene glycol (PEG), polyoxypropylene triol, polytetramethylene ether glycol (PTMEG), and the like. The polyester polyol is a polyol with repeating ester bonds (—COO—) and may include an adipic acid-based polyester polyol, a phthalic acid-based polyester polyol, a succinic acid-based polyester polyol, and the like.

In an embodiment, the composition for manufacturing polyurethane foam may include a polyether polyol and a polyester polyol in a weight ratio of 1:0.17 to 1:0.33. In other words, the polyurethane foam for cosmetic composition impregnation may be formed by mixing a polyether polyol and a polyester polyol in a weight ratio of 1:0.17 to 1:0.33 and foaming the mixture. In the present specification, the phrase “A and B are included/mixed and foamed in a weight ratio of X:Y” means that the “A:B weight ratio is X:Y.” Within the above range, the cosmetic impregnation performance and long-term stability of the polyurethane foam can be improved. For example, the weight ratio of a polyether polyol and a polyester polyol may be 1:0.17 or more, 1:0.19 or more, 1:0.21 or more, or 1:0.23 or more. For example, the weight ratio of a polyether polyol and a polyester polyol may be 1:0.33 or less, 1:0.31 or less, 1:0.29 or less, or 1:0.27 or less.

In an embodiment, the polyurethane foam for cosmetic composition impregnation may have a pore number per linear inch of 70 to 80 ppi. The pore number per linear inch is a value obtained by measuring the number of pores present per inch in the polyurethane foam, and it can be understood that as the value is higher, the polyurethane foam has a structure having many small pores, and as the value is lower, the polyurethane foam has a structure having large pores formed at wide intervals. When the pore number per inch falls within the above range, the cosmetic impregnation performance and long-term stability of the polyurethane foam can be improved over other polyurethane foams. For example, the pore number per linear inch of the polyurethane foam for cosmetic composition impregnation may be 70 ppi or more, 71 ppi or more, 72 ppi or more, 73 ppi or more, or 74 ppi or more. For example, the pore number per inch of the polyurethane foam for cosmetic composition impregnation may be 80 ppi or less, 79 ppi or less, 78 ppi or less, 77 ppi or less, or 76 ppi or less.

In an embodiment, the polyurethane foam for cosmetic composition impregnation may have a hardness of 70 to 85 on the Asker F hardness scale as measured by the Asker durometer Type F. The Asker durometer is a hardness measuring system used to measure the hardness of soft materials such as rubber, foam, and gel, and the Asker durometer Type F is mainly used to measure the hardness of soft foam materials such as polyurethane foam and sponges. When the Asker hardness falls within the above range, the cosmetic impregnation performance and long-term stability of the polyurethane foam can be improved. For example, the hardness of the polyurethane foam for cosmetic composition impregnation may be 70 or more, 72 or more, 74 or more, 76 or more, or 78 or more. For example, the hardness of the polyurethane foam for cosmetic composition impregnation may be 85 or less, 84 or less, 83 or less, 82 or less, or 81 or less.

In an embodiment, the polyurethane foam for cosmetic composition impregnation may have a tensile strength of 200 to 300 kPa. The tensile strength is a value indicating how much force a material can withstand before it breaks or fractures. When the tensile strength falls within the above range, the cosmetic impregnation performance and long-term stability of the polyurethane foam can be more favorable than for other polyurethane foams. For example, the tensile strength of the polyurethane foam for cosmetic composition impregnation may be 200 kPa or more, 210 kPa or more, 220 kPa or more, 230 kPa or more, or 240 kPa or more. For example, the tensile strength of the polyurethane foam for cosmetic composition impregnation may be 300 kPa or less, 290 kPa or less, 280 kPa or less, 270 kPa or less, 260 kPa or less, 250 kPa or less, or 240 kPa or less. For example, the tensile strength of the polyurethane foam for cosmetic composition impregnation may be 220 to 240 kPa.

In an embodiment, the polyurethane foam for cosmetic composition impregnation may have an elongation rate of 200 to 250%. The elongation rate is a value indicating that how much a material can stretch before it breaks and is expressed as a percentage. When the elongation rate falls within the above range, the cosmetic impregnation performance and long-term stability of the polyurethane foam can be more favorable than for other polyurethane foams. For example, the elongation rate of the polyurethane foam for cosmetic composition impregnation may be 200% or more, 210% or more, or 220% or more. For example, the elongation rate of the polyurethane foam for cosmetic composition impregnation may be 250% or less, 240% or less, or 230% or less. For example, the elongation rate of the polyurethane foam for cosmetic composition impregnation may be 210 to 250%.

In an embodiment, the polyurethane foam for cosmetic composition impregnation may be impregnated with a cosmetic composition having an oil-phase ingredient content of 25 wt % to 50 wt %. Here, the oil-phase ingredient may refer to a fat-soluble ingredient such as an oil (e.g., a vegetable oil, an animal oil, a mineral oil, a synthetic oil, etc.), a wax, a higher fatty acid, a higher alcohol, an ester-based oil, a silicone-based oil, an organic UV blocker, or the like. When the polyurethane foam is impregnated with a cosmetic composition having an oil-phase ingredient content of 25 wt % to 50 wt %, excellent impregnation performance can be exhibited. For example, the oil-phase ingredient content of a cosmetic composition may be 25 wt % or more, 30 wt % or more, 35 wt % or more, 40 wt % or more, or 45 wt % or more. For example, the oil-phase ingredient content of a cosmetic composition may be 50 wt % or less, 45 wt % or less, 40 wt % or less, 35 wt % or less, or 30 wt % or less.

In an embodiment, the polyurethane foam for cosmetic composition impregnation may be impregnated with a cosmetic composition having a viscosity of 5,000 cps or more and 45,000 cps or less. For example, the viscosity of the impregnated cosmetic composition may be equal to or greater than 5,000 cps, 7,000 cps, 10,000 cps, 15,000 cps, 20,000 cps, 25,000 cps, or 30,000 cps, or equal to or less than 45,000 cps, 40,000 cps, 37,000 cps, 35,000 cps, 30,000 cps, 25,000 cps, or 20,000 cps. For example, the viscosity of the cosmetic composition may be 7,000 cps or more and 45,000 cps or less. For example, the viscosity of the cosmetic composition may be 7,000 cps or more and 40,000 cps or less. For example, the viscosity of the cosmetic composition may be 10,000 cps or more and less than 40,000 cps.

In an embodiment, the cosmetic composition may include an additive typically used in the art within a wt % of composition range that does not interfere with the effectiveness of the polyurethane foams described herein for cosmetic composition impregnation and application of the cosmetic composition to human skin. For example, the cosmetic composition may further include a thickener, a pH adjusting agent, a surfactant, a stabilizer, a preservative, an antiseptic, a bactericide, a moisturizer, a sunscreen, a skin conditioning agent, a whitening agent, a colorant, an organic pigment, an inorganic pigment, a fragrance, a vitamin, a natural pigment, a pearl, other functional ingredients, and the like. Here, the functional ingredient may include, for example, a wrinkle improving ingredient, a whitening ingredient, an antioxidant ingredient, an anti-aging ingredient, a plumping ingredient, an exfoliating ingredient, a scrubbing ingredient, a moisturizing ingredient, a skin barrier strengthening ingredient, a skin elasticity strengthening ingredient, and the like. The blending amount of the ingredients may be easily selected by those skilled in the art to be within a range that does not impair the purpose and effects of the polyurethane foams described herein.

In an embodiment, the cosmetic composition may be provided in a formulation typically prepared in the art. For example, the cosmetic composition may be formulated into an oil dispersion formulation in which a powder complex is dispersed in an oil phase, an aqueous dispersion formulation in which a powder complex is dispersed in an aqueous phase, an emulsion formulation of oil-in-water (O/W), water-in-oil (W/O), water-in-oil-in-water (W/O/W), or oil-in-water-in-oil (O/W/O) type, a solubilized formulation, or the like. However, the present invention is not limited thereto, and the cosmetic composition may also be provided in a formulation such as a solution, a suspension, an emulsion, or the like.

In an embodiment, the cosmetic composition may be prepared in the form of various products. For example, it may be prepared as a sun care product such as a sunscreen, sun milk, sun lotion, or sun cushion. Alternatively, it may be prepared as a soap, detergent, or cleansing cream or as a color cosmetic such as a lipstick, makeup base, foundation, compact, concealer, or skin cover. Also, it may be prepared as a hair cleansing product such as a shampoo, rinse, hair conditioner, or hair gel.

In an embodiment, the composition may be a composition for an external preparation for skin. For example, the composition for an external preparation for skin may have a formulation such as a solution or the like.

In an embodiment, the composition for an external preparation for skin may include an ingredient used in an external preparation for skin of typical cosmetics or drugs, for example, an aqueous-phase ingredient, an oil-phase ingredient, a powdered ingredient, an alcohol, a moisturizer, a thickener, a sunscreen, a whitening agent, an antiseptic, an antioxidant, a surfactant, a fragrance, a coloring agent, a fatty substance, a solubilizer, a thickening agent, a gelling agent, a softener, a foaming agent, an air freshener, a metal ion sequestering agent, a chelating agent, a vitamin, various skin nutrients, or a combination thereof, which may be appropriately blended as necessary. The blending amount of the ingredients may be easily selected by those skilled in the art within the range that does not impair the purpose and effects of the polyurethane foams described herein.

According to another aspect of the present application, there is provided a method of manufacturing polyurethane foam for cosmetic composition impregnation. The method of manufacturing polyurethane foam for cosmetic composition impregnation includes mixing a composition for manufacturing polyurethane foam; and foaming the mixed polyurethane foam composition. The detailed description of a polyurethane foam for cosmetic composition impregnation manufactured by the above method is as described above.

In an embodiment, the composition for manufacturing polyurethane foam may include a polyether polyol and a polyester polyol. The detailed descriptions of the polyether polyol, the polyester polyol, and the mixing ratio thereof are as described above.

In an embodiment, the composition for manufacturing polyurethane foam may include a polyether polyol, a polyester polyol, a foaming agent, a catalyst, and an additive. The foaming agent may form air bubbles in the polyurethane foam to form a porous structure and may include, for example, water, hydrochlorofluorocarbon-141b (HCFC-141b), hydrofluorocarbon-245fa (HFC-245fa), and the like. The catalyst may control the curing time, mechanical properties, and final structure of the polyurethane foam and may include, for example, dimethylethanolamine (DMEA), dibutyltin dilaurate (DBTDL), triethylenediamine (TEDA), 1,4-diazabicyclo[2.2.2]octane (DABCO), and the like. The additive may improve the physical and chemical properties of polyurethane foam or may impart a specific function to polyurethane foam and may include, for example, a silicone surfactant, tris(1-chloro-2-propyl) phosphate (TCPP), calcium carbonate, and the like. When the type and content of foaming agent, catalyst, and additive are appropriately adjusted, the physical properties of the polyurethane foam can be optimized.

In an embodiment, the composition for manufacturing polyurethane foam may further include a reactant. The reactant may be, for example, a material with two or more isocyanate groups. For example, the reactant may be a diisocyanate. For example, the reactant may include toluene diisocyanate (TDI), diphenylmethane diisocyanate (MDI), naphthalene diisocyanate (NDI), hexamethylene diisocyanate (HDI), and the like.

In an embodiment, the mixing of a composition for manufacturing polyurethane foam may be performed at a temperature of 18 to 28° C. and a stirring speed of 4,000 to 5,000 rpm for 20 to 40 minutes. Under these conditions, polyurethane foam physical properties favorable for use of the polyurethane foam for cosmetic composition impregnation according to the present invention can be achieved.

In an embodiment, in the foaming, the polyether polyol and the polyester polyol may react with the diisocyanate to form polyurethane foam.

In an embodiment, the foaming may be performed at a discharge pressure of 35,000 to 45,000 Pa. Under these conditions, polyurethane foam physical properties favorable for use of the polyurethane foam for cosmetic composition impregnation according to the present invention can be achieved.

Hereinafter, the present invention will be described with reference to manufacturing examples and experimental examples, and the scope of the present invention is not limited to the following examples.

### MANUFACTURING EXAMPLES

### (1) Manufacture of Manufacturing Examples 1 to 3

Polyol compositions according to the contents shown in Table 1 were each mixed with methylene diphenylmethane diisocyanate (MDI) in a weight ratio of 1:1 at a temperature of 18 to 28° C. and a stirring speed of 4,500 rpm for 30 minutes, and then the resulting mixture was foamed at a discharge pressure of 40,000 Pa to manufacture the polyurethane foams of Manufacturing Examples 1 to 3. The content units in Table 1 are wt %.

TABLE 1

| | | Manufacturing | Manufacturing | Manufacturing |
|---|---|---|---|---|
| Classification | | Example 1 | Example 2 | Example 3 |
| Polyol | Polyether polyol | 78.1 | 68.1 | — |
| composition | Polyester polyol | 19.2 | 29.2 | 97.3 |
| | Others | 2.7 | 2.7 | 2.7 |

| | | | |
|---|---|---|---|
| Total | 100 | 100 | 100 |

### (2) Manufacture of Manufacturing Examples 4 and 5

The physical properties of the polyurethane foam manufactured according to Manufacturing Example 1 are shown in Table 2. Also, polyurethane foams of Manufacturing Examples 4 and 5 were manufactured by varying the physical properties of polyurethane foam as shown in Table 2 while changing the conditions for the process of manufacturing polyurethane foam.

The pore number per linear inch (ppi) of Manufacturing Examples 1, 4, and 5 was measured in accordance with ASTM WI-QA-14, the Asker hardness on the Asker F hardness scale was measured in accordance with JSI K6301A, the elongation rate was measured in accordance with ASTM D638, and the tensile strength was measured in accordance with ISO 1798.

TABLE 2

| | Manufacturing | Manufacturing | Manufacturing |
|---|---|---|---|
| | Example 1 | Example 4 | Example 5 |
| Pore number per inch | 75 | 45 | 100 |
| (ppi) | | | |
| Asker (type F) | 80 | 50 | 90 |
| hardness | | | |
| Elongation rate (%) | 232 | 263 | 208 |
| Tensile strength (kPa) | 237 | 245 | 212 |

### EXPERIMENTAL EXAMPLES

### (1) Experimental Example 1: Evaluation of Stability of Polyurethane Foam

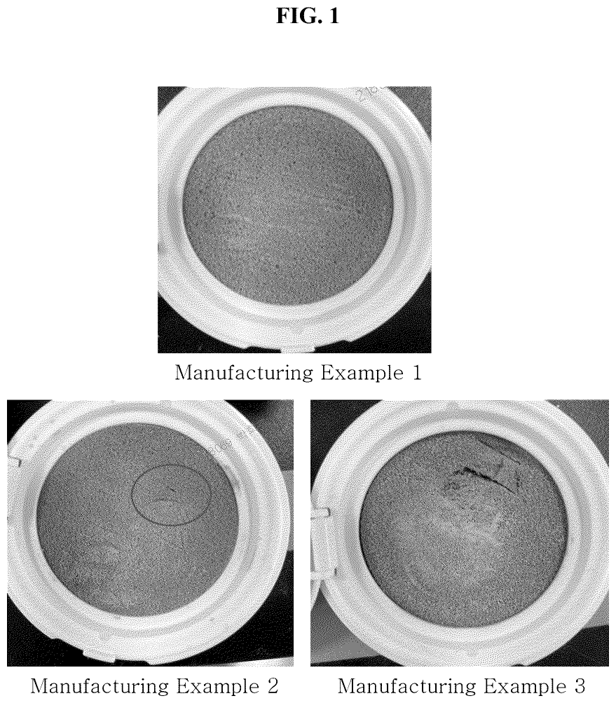

Each polyurethane foam of Manufacturing Examples 1 to 3 was input into an incubator set at a temperature of 70° C. and a humidity of 90%, whether hydrolysis occurred and whether elastic recovery was maintained were visually observed over time, and results are shown in Table 3. Here, whether elastic recovery was maintained was evaluated by pressing each polyurethane foam with a sharp object for 1 second and then determining whether tearing occurred. Also, photographs taken after each polyurethane foam of Manufacturing Examples 1 to 3 was pressed with a sharp object for 1 second after 3 months of being input in the incubator are shown in FIG. 1.

TABLE 3

| | After 1 | After 2 | After 1 | After 2 | After 3 |
|---|---|---|---|---|---|
| | day | weeks | month | months | months |
| Manufacturing | — | — | — | — | Elastic recovery |
| Example 1 | | | | | was maintained |
| Manufacturing | — | — | — | — | Elastic recovery |
| Example 2 | | | | | was degraded |
| Manufacturing | — | Hydrolyzed | Hydrolyzed | Hydrolyzed | Hydrolyzed |
| Example 3 | | | | | |

As experimental results, in the case of the polyurethane foam of Manufacturing Example 1, it can be confirmed that hydrolysis did not occur and elastic recovery was maintained even after 3 months.

On the other hand, in the case of Manufacturing Example 2, it was confirmed that elastic recovery was degraded after 3 months. In the case of Manufacturing Example 3, it was confirmed that hydrolysis had occurred after 2 weeks.

### (2) Experimental Example 2: Evaluation of Cosmetic Composition Impregnation Performance of Polyurethane Foam

The cosmetic impregnation performance of Manufacturing Examples 1, 4, and 5 was evaluated in terms of fillability, loadability, and dischargeability, and results are shown in Table 4. A case in which fillability, loadability, or dischargeability was excellent was evaluated as O, and a case in which each item was poor was evaluated as X.

Specifically, in terms of fillability, 10 g of a cosmetic composition having an oil-phase ingredient content of 40% and a viscosity of 20,000 cps was applied onto the polyurethane foam, and 1 day later, whether the cosmetic composition was deposited inside the polyurethane foam was confirmed. In this regard, a case in which the cosmetic composition was completely deposited was evaluated as excellent fillability, and a case in which the cosmetic composition remained undeposited was evaluated as poor fillability. Unless otherwise indicated, viscosities reported in the present disclosure were measured using a Brookfield DVplus Viscometer, Spindle No. 4, at 6 rpm and 25° C.

To evaluate loadability, 10 g of a cosmetic composition having an oil-phase ingredient content of 40% and a viscosity of 20,000 cps was applied onto the polyurethane foam, and the change in weight of the polyurethane foam before and after impregnation was measured to determine the total amount (g) of the cosmetic composition loaded. A case in which the loading amount was 9 g or more was evaluated as excellent loadability, and a case in which the loading amount was less than 9 g was evaluated as poor loadability.

Additionally, in terms of dischargeability, the polyurethane foam impregnated with the cosmetic composition was pressed with a pressure of 100 N/m² using a cosmetic puff to apply the cosmetic composition onto the cosmetic puff, and the change in weight of the cosmetic puff before and after application was measured to determine the total amount (g) of the cosmetic composition discharged. A case in which the discharge amount was 0.2 to 0.4 g was evaluated as excellent dischargeability, a case in which the discharge amount was less than 0.2 g was evaluated as poor dischargeability, and a case in which the discharge amount exceeds 0.4 g was evaluated as poor dischargeability because use of the polyurethane foam applicator was difficult due to an excessively large discharge amount and because the cosmetic flowed out of the polyurethane foam even without an external force.

TABLE 4

| Classification | Fillability | Loadability | Dischargeability |
|---|---|---|---|
| Manufacturing | ◯ | ◯ | ◯ |
| Example 1 | (completely | (9.8 g) | (0.25 g) |
| | deposited | | |
| | after 1 day) | | |
| Manufacturing | ◯ | ◯ | X |
| Example 4 | (completely | (9.8 g) | (0.45 g) |
| | deposited | | |
| | after 1 day) | | |
| Manufacturing | X | X | X |
| Example 5 | (cosmetic | (loading | (0.10 g) |
| | composition | was not | |
| | remained on | possible) | |
| | the surface | | |
| | after 1 day) | | |

As experimental results, in the case of Manufacturing Example 1, it can be confirmed that fillability, loadability, and dischargeability were all excellent, and thus it was suitable for cosmetic composition impregnation.

On the other hand, in the case of Manufacturing Example 4, it can be confirmed that its use was difficult due to an excessively large cosmetic discharge amount, and the cosmetic flowed out of the polyurethane foam even without an external force, thereby exhibiting poor dischargeability. In the case of Manufacturing Example 5, it can be confirmed that fillability, loadability, and dischargeability were all overall poor.

### (3) Experimental Example 3: Evaluation of Impregnation Performance According to Oil-Phase Ingredient Content and Viscosity of Cosmetic Composition

The cosmetic impregnation performance of the polyurethane foam of Manufacturing Example 1 was evaluated in terms of fillability, loadability, and dischargeability by varying the oil-phase ingredient content and viscosity of an impregnated cosmetic composition as shown in Table 5, and results are shown in Table 5. Fillability, loadability, and dischargeability were evaluated by the same method as in Experimental Example 2.

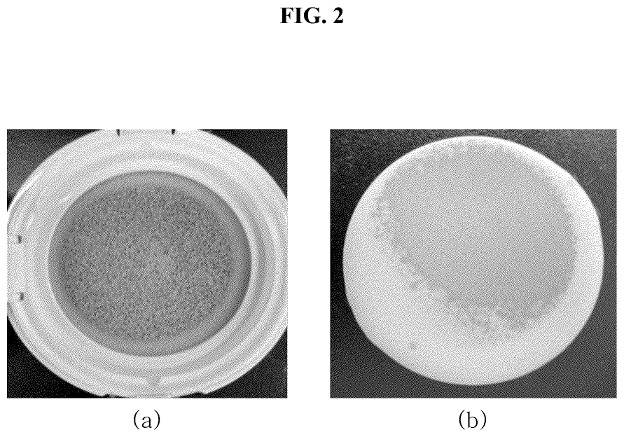

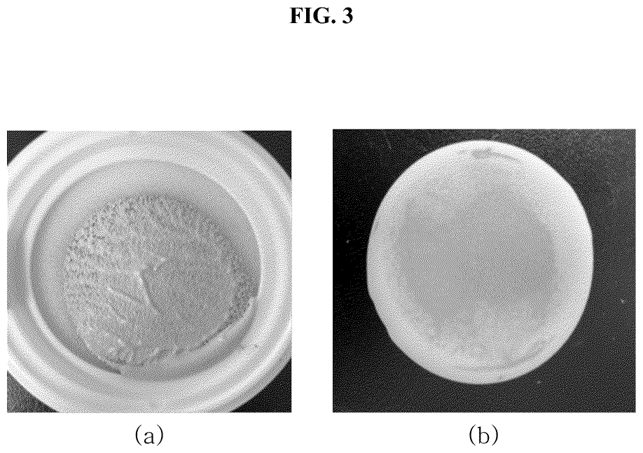

A photograph of the polyurethane foam of Manufacturing Example 1 filled with a cosmetic composition having an oil-phase ingredient content of 40 wt % and a viscosity of 20,000 cps is shown in FIG. 2(a), and a photograph of a cosmetic puff to which the cosmetic composition was applied by pressing the filled polyurethane foam is shown in FIG. 2(b). Also, a photograph of the polyurethane foam of Manufacturing Example 1 filled with a cosmetic composition having an oil-phase ingredient content of 20 wt % and a viscosity of 50,000 cps is shown in FIG. 3(a), and a photograph of a cosmetic puff to which the cosmetic composition was applied by pressing the filled polyurethane foam is shown in FIG. 3(b).

TABLE 5

| Classification | | | | |
|---|---|---|---|---|
| Oil-phase | | | | |
| ingredient | | | | |
| content | Viscosity | Impregnation performance | | |
| (wt %) | (cps) | Fillability | Loadability | Dischargeability |
| 20 | 50,000 | X | X | X |
| | | (cosmetic | (8.0 g) | (0.12 g) |
| | | composition | | |
| | | remained on | | |
| | | the surface | | |
| | | after 1 day) | | |
| 25 | 35,000 | ◯ | ◯ | ◯ |
| | | (completely | (9.8 g) | (0.22 g) |
| | | deposited | | |
| | | after 1 day) | | |
| 40 | 20,000 | ◯ | ◯ | ◯ |
| | | (completely | (9.8 g) | (0.25 g) |
| | | deposited | | |
| | | after 1 day) | | |
| 50 | 10,000 | ◯ | ◯ | ◯ |
| | | (completely | (9.8 g) | (0.25 g) |
| | | deposited | | |
| | | after 1 day) | | |
| 60 | 3,000 | ◯ | ◯ | X |
| | | (completely | (9.9 g) | (0.48 g) |
| | | deposited | | |
| | | after 1 day) | | |

As experimental results, in the case of the polyurethane foam of Manufacturing Example 1, it can be confirmed that fillability, loadability, and dischargeability with respect to the cosmetic compositions having an oil-phase ingredient content of 25 to 50 wt % and a viscosity of 5,000 cps or more and 45,000 or less were all excellent, and thus it was suitable for cosmetic composition impregnation.

Embodiments of the present invention provide polyurethane foams for cosmetic composition impregnation, wherein the foams exhibit improved long-term stability, can be easily filled and loaded with a cosmetic composition, and can easily discharge the cosmetic composition.

According to embodiments of the present invention, a polyurethane foam, which is impregnated with a cosmetic composition having an oil-phase ingredient content of 25 wt % to 50 wt % and/or a viscosity of 5,000 cps or more and 45,000 cps or less, with high long-term stability and impregnation performance, can be provided.

However, the effects obtainable in embodiments of the present application are not limited to the above-described effects, and other effects which are not described herein will be fully understood by those skilled in the art from the above-described descriptions.

Although certain embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

What is claimed is:

1. A polyurethane foam for cosmetic composition impregnation, the foam being formed by mixing a polyether polyol and a polyester polyol in a weight ratio of 1:0.17 to 1:0.33 and foaming the mixture.

2. The polyurethane foam of claim 1, the foam having a pore number per linear inch of 70 to 80 ppi and a hardness of 70 to 85 on the Asker F hardness scale as measured by the Asker durometer Type F.

3. The polyurethane foam of claim 1, the foam having a tensile strength of 220 to 240 kPa and an elongation rate of 210 to 250%.

4. The polyurethane foam of claim 1, wherein the foam is impregnated with a cosmetic composition having an oil-phase ingredient content of 25 wt % to 50 wt %.

5. The polyurethane foam of claim 1, wherein the foam is impregnated with a cosmetic composition having a viscosity of 7,000 cps or more and 45,000 cps or less.

6. A method of manufacturing polyurethane foam for cosmetic composition impregnation, comprising:
mixing a composition for manufacturing polyurethane foam, the composition including a polyether polyol and a polyester polyol; and
foaming the composition,
wherein the polyether polyol and the polyester polyol are mixed in a weight ratio of 1:0.17 to 1:0.33 in the composition for manufacturing polyurethane foam.

7. The method of claim 6, wherein the polyurethane foam for cosmetic composition impregnation has a pore number per linear inch of 70 to 80 ppi and a hardness of 70 to 85 on the Asker F hardness scale as measured by the Asker durometer Type F.

8. The method of claim 6, wherein the polyurethane foam for cosmetic composition impregnation has a tensile strength of 220 to 240 kPa and an elongation rate of 210 to 250%.

9. The method of claim 6, further comprising impregnating the polyurethane foam for cosmetic composition impregnation with a cosmetic composition having an oil-phase ingredient content of 25 wt % to 50 wt %.

10. The method of claim 6, further comprising impregnating the polyurethane foam for cosmetic composition impregnation with a cosmetic composition having a viscosity of 7,000 cps or more and 45,000 cps or less.

11. The method of claim 6, wherein the composition for manufacturing polyurethane foam includes the polyether polyol, the polyester polyol, a foaming agent, a catalyst, and an additive.

12. The method of claim 6, wherein the mixing of a composition for manufacturing polyurethane foam is performed at a temperature of 18 to 28° C. and a stirring speed of 4,000 to 5,000 rpm for 20 to 40 minutes.

13. The method of claim 6, wherein the foaming is performed at a discharge pressure of 35,000 to 45,000 Pa.
